(19) United States
(12) Patent Application Publication
(10) Pub. No.: US 2008/0096971 A1
(43) Pub. Date: Apr. 24, 2008

(54) Treatment of Inflammatory Disorders and Pain

(76) Inventors: Andrew Douglas Baxter, Cambridge (GB); John Brew, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/662,115

(22) PCT Filed: Sep. 7, 2005
(86) PCT No.: PCT/GB05/03452

§ 371 Date: Jun. 5, 2007

(30) Foreign Application Priority Data
Sep. 7, 2004 (GB) 0419828.9
Oct. 27, 2004 (GB) 0423926.5

(51) Int. Cl. A61K 31/135; A61P 1/00; A61P 29/00

(52) U.S. Cl. 514/646

(57) ABSTRACT

Compounds that may be used for the treatment or prevention of a condition associated with T-cell proliferation or that is mediated by pro-inflammatory cytokines are of formula (I): 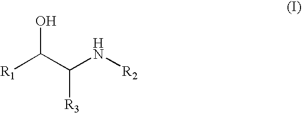
wherein R₁ is CHR₄—OR₅ or CHR₄—SR₅, or aryl or heteroaryl optionally substituted with one or more groups R₆; R₂ is alkyl or is part of a ring with R₃; R₃ is H, alkyl or CH₂ (when forming part of a ring with R₂); R₄ is H or alkyl or is part of a ring with R₅; R₅ is aryl or heteroaryl optionally substituted with R₇; each R₆ is independently alkyl, CF₃, OH, Oalkyl, OCOalkyl, CONH₂, CN, halogen, NH₂, NO₂, NHCHO, NHCONH₂, NHSO₂alkyl, CONH₂, SOMe, SO₂NH₂, Salkyl, CH₂SO₂alkyl or OCONalkyl₂; R₇ is R₈ or (CH₂)ₙOR₈, R₉, CF₃, OH, OR₉, OCOR₉, COR₉, COOR₉, CONH₂, CH₂CONH₂, CN, halogen, NH₂, NO₂, NHCHO, NHCONH₂, NHCONHR₇, NHCON(R₉)₂, NHCOR₉, NHCOaryl, NHSO₂Me, CONH₂, SMe, SOMe Or SO₂NH₂; R₈ is (CH₂)ₙOR₉, (CH₂)ₙOR₉, (CH₂)ₙCOOR₉ or (CH₂)ₙCOaryl; R₉ is alkyl or cycloalkyl; and n is 1 to 4; or a salt thereof.

### FIELD OF THE INVENTION

This invention relates to the treatment of inflammatory disorders and pain

### BACKGROUND OF THE INVENTION

Immune-driven inflammatory events are a significant cause of many chronic inflammatory diseases where prolonged inflammation causes tissue destruction and results in extensive damage and eventual failure of the effected organ. The cause of these diseases is unknown, so they are often called autoimmune, as they appear to originate from an individual's immune system turning on itself. Conditions include those involving multiple organs, such as systemic lupus erythematosus (SLE) and scleroderma. Other types of autoimmune disease can involve specific tissues or organs such as the musculoskeletal tissue (rheumatoid arthritis, ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy, interstitial nephritis), exocrine glands (Sjogren's syndrome and autoimmune pancreatitis) and skin (psoriasis and atopic dermatitis).

In addition, there are chronic inflammatory diseases whose aetiology is more or less known but whose inflammation is also chronic and unremitting. These also exhibit massive tissue/organ destruction and include conditions such as osteoarthritis, periodontal disease, diabetic nephropathy, chronic obstructive pulmonary disease, artherosclerosis, graft versus host disease, chronic pelvic inflammatory disease, endometriosis, chronic hepatitis and tuberculosis. In these diseases, the tissue destruction often damages organ function, resulting in progressive reductions in quality of life and organ failure. These conditions are a major cause of illness in the developing world and are poorly treated by current therapies.

Inflammation of skin structures (dermatitis) is a common set of conditions which include actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. These diseases are treated using a wide array of therapies, many of which have very severe side-effects.

Current disease-modifying treatments (if any) for immune-driven conditions include neutralising antibodies, cytotoxics, corticosteroids, immunosuppressants, antihistamines and antimuscarinics. These treatments are often associated with inconvenient routes of administration and severe side-effects, leading to compliance issues. Moreover, certain drug classes are only effective for certain types of inflammatory diseases, e.g. antihistamines for rhinitis.

Beta-amino alcohols are known to have antihypertensive, vasodilator, sympathomimetic, bronchodilator or cardiostimulant activity through agonism and antagonism at alpha and beta adrenoceptors.

### SUMMARY OF THE INVENTION

Surprisingly, it has been found that beta-amino alcohols (I) are inhibitors of cytokines and possess anti-inflammatory properties. According to the present invention, pain or an inflammatory condition, e.g. described above, is treated by the use of a compound of general formula (I) 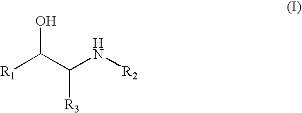
wherein

R₁ is CHR₄—OR₅ or CHR₄—SR₅, or aryl or heteroaryl optionally substituted with one or more groups R₆;

R₂ is alkyl or is part of a ring with R₃;

R₃ is H, alkyl or CH₂ (when forming part of a ring with R₂);

R₄ is H or alkyl or is part of a ring with R₅;

R₅ is aryl or heteroaryl optionally substituted with R₇;

each R₆ is independently alkyl, CF₃, OH, Oalkyl, OCOalkyl, CONH₂, CN, halogen, NH₂, NO₂, NHCHO, NHCONH₂, NHSO₂alkyl, CONH₂, SOMe, SO₂NH₂, Salkyl, CH₂SO₂alkyl or OCONalkyl₂;

R₇ is R₈ or (CH₂)ₙOR₈, R₉, CF₃, OH, OR₉, OCOR₉, COR₉, COOR₉, CONH₂, CH₂CONH₂, CN, halogen, NH₂, NO₂, NHCHO, NHCONH₂, NHCONHR₇, NHCON(R₉)₂, NHCOR₉, NHCOaryl, NHSO₂Me, CONH₂, SMe, SOMe or SO₂NH₂;

R₈ is (CH₂)ₙOR₉, (CH)ₙOR₉, (CH₂)ₙCOOR₉ or (CH₂)ₙCOaryl;

R₉ is alkyl or cycloalkyl; and

n is 1 to 4;

or a salt thereof.

### DESCRIPTION OF THE INVENTION

Compounds of formula (I) include those wherein R₁ is aryl or heteroaryl and those wherein R₁ is CHR₄—OR₅. Specific compounds for use in the invention include albuterol, amidephrine, amiterol, arotinol, bambuterol, bamethan, bronkosol, bucumolol, butidrine, butoxamine, carbuterol, cimaterol, clenbuterol, clorprenaline, colterol, deterenol, diacetylisoproterenol, dichloroisoproterenol, dioxifedrine, dimetofrine, dipivefrin, divabuterol, epinephrine, ephedrine, etilefrine, fenoterol, flerobuterol, halostachine, ibuterol, isoetharine, isoprenaline, isopropylmethoxamine, isoproterenol, mabuterol, meluadrine, menetyl, metalol, metaproterenol, metaterol, metiprenaline, nifenalol, oxedrine, oxilofrine, phenylephrine, procaterol, pronetalol, pseudoephedrine, quinterenol, rimiterol, salbutamol, sotalol, soterenol, sulfonterol, suloctidil, sympatol, terbutaline and tulobuterol; alprenolol, atenolol, befunolol, betaxolol, bunitrolol, bunolol, bupranolol, carteolol, cloranolol, esmolol, exaprolol, mepindolol, metipranolol, metoprolol, moprolol, nadolol, nitrolol, oxprenolol, penbutolol, pindolol, practolol, prenalterol, propafenone, propanolol, ridazolol, tertalolol, tiprenolol, talinolol, tilisodol and vanilol. Preferred compounds are clenbuterol, mabuterol, procaterol and rimiterol.

It is understood that compounds for use in the invention include salts, e.g. the hydrochloride, metabolites and pro-drugs thereof. Compounds for use in the invention may be chiral, and it will be understood that this invention includes any diastereomers and enantiomers of (I).

A preferred diastereomer or enantiomer of (I) has little or no activity at the α or β adrenoceptors. This activity may be determined by use of the appropriate in vitro assay. Particularly preferred compounds include (S)-(+)-clenbuterol, (S)-(+)-mabuterol, erythro-(S)-3,4-dihydroxyphenyl-(R)-piperidin-2-yl-methanol, threo-(S)-3,4-dihydroxyphenyl-(S)-piperidin-2-yl-methanol and erythro-(S)-3,5-dichloro-4-aminophenyl-(R)-piperidin-2-yl methanol.

The compounds of formula (I) according to the invention are used to treat inflammatory diseases including, but not exclusive to, autoimmune diseases involving multiple organs, such as systemic lupus erythematosus (SLE) and scleroderma, specific tissues or organs such as the musculoskeletal tissue (rheumatoid arthritis, ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy, interstitial nephritis) exocrine glands (Sjogren's syndrome and autoimmune pancreatitis) and skin (psoriasis and atopic dermatitis), chronic inflammatory diseases such as osteoarthritis, periodontal disease, diabetic nephropathy, chronic obstructive pulmonary disease, artherosclerosis, graft versus host disease, chronic pelvic inflammatory disease, endometriosis, chronic hepatitis and tuberculosis, IgE mediated (Type I) hypersensitivities such as rhinitis, asthma, anaphylaxis and dermatitis. Dermatitis conditions include; actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria. Conditions of the eye, such as diabetic retinopathy, macular degeneration, uveitis and conjunctivitis, may also be treated.

These compounds may be used according to the invention when the patient is also administered or in combination with another therapeutic agent selected from corticosteroids (examples including cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone), disease modifying anti-rheumatic drugs (DMARDs) (examples including azulfidine, aurothiomalate, bucillamine, chlorambucil, cyclophosphamide, leflunomide, methotrexate, mizoribine, penicillamine and sulphasalazine), immunosuppressants (examples including azathioprine, cyclosporin, mycophenolate), COX inhibitors (examples including aceclofenac, acemetacin, alcofenac, alminoprofen, aloxipirin, amfenac, aminophenazone, antraphenine, aspirin, azapropazone, benorilate, benoxaprofen, benzydamine, butibufen, celecoxib, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, mefenamic acid, metamizole, mofebutazone, mofezolac, nabumetone, naproxen, nifenazone, oxametacin, phenacetin, pipebuzone, pranoprofen, propyphenazone, proquazone, rofecoxib, salicylamide, salsalate, sulindac, suprofen, tiaramide, tinoridine, tolfenamic acid, zomepirac) neutralising antibodies (examples including etanercept and infliximab), antibiotics (examples including doxycycline and minocycline).

According to another aspect of the invention, the compounds of formula (I) exhibit analgesic activity in animal models. The activity of these compounds may be determined by the use of the appropriate in vivo assay.

This invention also relates to a method of treatment for patients (including man and/or mammalian animals raised in the dairy, meat or fur industries or as pets) suffering from chronic, acute or neuropathic pain; and more specifically, a method of treatment involving the administration of the analgesic of formula (I) as the active constituent.

Accordingly, the compounds of formula (I) can be used inter alia in the treatment of pain conditions such as acute and chronic pain (as well as, but not limited to, pain associated with cancer, surgery, arthritis, dental surgery, trauma, musculo-skeletal injury or disease, visceral diseases) and migraine headache. Additionally the painful conditions can be neuropathic; examples of such conditions are post-herpetic neuralgia, diabetic neuropathy, drug-induced neuropathy, HIV-mediated neuropathy, sympathetic reflex dystrophy or causalgia, fibromyalgia, myofacial pain, entrapment neuropathy, phantom limb pain and trigeminal neuralgia. Neuropathic conditions include central pain related to stroke, multiple sclerosis, spinal cord injury, arachnoiditis, neoplasms, syringomyelia, Parkinson's disease and epilepsia.

It will often be advantageous to use compounds of formula (I) in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

Any suitable route of administration can be used. For example, any of oral, topical, parenteral, ocular, rectal, vaginal, inhalation, buccal, sublingual and intranasal delivery routes may be suitable. The dose of the active agent will depend on the nature and degree of the condition, the age and condition of the patient and other factors known to those skilled in the art. A typical dose is from 0.1, e.g. 10 to 100, mg given one to three times per day.

The following studies provide evidence on which the present invention is based.

Beta2 Agonism Functional Assay

Guinea-pig trachea ring preparations were suspended in Kreb's solution containing indomethacin. After 15 minutes stabilisation, the preparations were repeated contracted using carbachol and simultaneously treated with increasing cumulative doses test compounds (0.1 nM to 0.1 μM). Beta2 agonism for each test compound was determined by its dose-dependant inhibition of carbachol-stimulated tracheal muscle twitch.

(S)-Clenbuterol caused up to 75% inhibition at from −10 to −7 log M, a level a little lower than that caused by formoterol (positive control). (R)-Clenbuterol showed little such beta2 agonist activity.

LPS Mouse Assay

7 week old Balb C ByJ mice (24-28 g) were administered, either by i.p. (5 ml/kg) or oral (10 ml/kg) administration, with vehicle or test article. 30 minutes later these animals were challenged with an intraperitoneal injection of 1 mg/kg LPS. 2 hours after LPS challenge blood samples were collected under light isoflurane anaesthesia into normal tubes by retro-orbital puncture. Samples were allowed to clot at room temperature and then spun at 6000 g for 3 min at 4° C. Serum was stored at −20° C. until use. Serum TNFα and IL-10 levels were analysed in duplicate by ELISA technique.

(S) and (R)-Clenbuterol each exhibited significant activity at doses, i.e. 0.3 and 1 mg/kg p.o. (for the (S)-clenbuterol enantiomer), that produce plasma loads below beta2 agonist activity concentrations. (S)-clenbuterol at a dose that would have not caused beta2 agonism had a profound immunomodulatory profile. This immunomodulatory effect is less than (R)-clenbuterol which has distinct beta2 agonism, a pharmacology which has known anti-inflammatory profile. However the effect is distinct and should have strong anti-inflammatory activity through a seemingly previously unappreciated pharmacodynamic effect.

Carrageenan Paw Assay

Fasted (18 hour) male Wistar rats (105-130 g) were weighed and a basal mercury plethysmometer reading was taken of the right hind paw by submerging the paw in the mercury up to the tibiotarsal joint. Subsequently, vehicles, reference items and test articles were administered by oral gavage (10 ml/kg). Half an hour after treatment 0.1 ml of 2% carrageenan in 0.9% saline was injected into the subplanatar area of the right hind paw. The right paw was measured again with the plethysmometer at 1, 2, 3, 4 and 5 hours after carrageenan administration.

(S)-Clenbuterol had a strong and dose-dependent anti-inflammatory activity which is highly unlikely to involve beta2 agonism, at doses of 0.3, 1 and 3 mg/kg oral. The change in pore volume over time at 0.3 mg/kg oral was similar to that observed for ibuprofen at 100 mg/kg.

Rat Adjuvant Assay

Male Wistar rats (180 to 200 g) were inoculated by subplantar injection of Freund's adjuvant (suspension of Mycobacterium butyricum in mineral oil) into the right paw at day 0. Sham inoculations were injected in the same way with 0.9% saline in matched Male Wistar rats. On day 2 animals were weighed. On days 3, 4, 7, 9 and 11 animals were weighed and both their right and left hind paws were measure by plethsymometry by submerging the paw up to the tibiotarsal joint. On day 11, rats with left hind paw volumes increased by 20% were selected for continuance in the study. On the same day continuance rats were administered test article orally (10 ml/kg in distilled water) and from then on once a day until the completion of the study. Left and right hind paw volumes were measured on days 11, 14, 15, 16, 18 and 21.

(S)-Clenbuterol exhibited a clear but non-dose-related anti-inflammatory effect in this model, at doses of 0.3, 1 and 3 mg/kg/day. (R)-Atenolol showed a similar effect.

Collagen II-Induced Arthritis Assay

6 to 8 week old male DBA1 mice were immunised with 0.1 ml intradermal (tail) injection of bovine type II collagen emulsified in Freund's complete adjuvant (day 0). At day 21 a booster collagen (intraperitoneal) inoculation was given, in PBS. Simultaneously, administration of test compound was given by the predetermined route of administration and delivery frequency. In addition, day 21 commenced observation of clinical signs, body weights and arthritis reaction scores. Arthritis score was assessed by the summation of all four paws scored for signs of arthritis development.

(S)-Clenbuterol exhibited a clear but inverse dose-related anti-inflammatory effect in the collagen II induced arthritis model in the mouse, at doses of 0.3, 1 and 3 mg/kg.

In summary, (S)-clenbuterol has been shown to have a clear anti-inflammatory effect which is unrelated to its racemate's beta2 agonist activity. Also this anti-inflammatory activity has been observed across a number of inflammatory assays, suggesting a potentially broad therapeutic utility.

1. A method for the treatment or prevention of a condition associated with T-cell proliferation or that is mediated by pro-inflammatory cytokines, wherein said method comprises administering, to a patient in need of such treatment or prevention, a compound of formula (I) 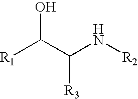
wherein
R₁ is CHR₄—OR₅ or CHR₄—SR₅, or aryl or heteroaryl optionally substituted with one or more groups R₆;
R₂ is alkyl or is part of a ring with R₃;
R₃ is H, alkyl or CH₂ (when forming part of a ring with R₂);
R₄ is H or alkyl or is part of a ring with R₅;
R₅ is aryl or heteroaryl optionally substituted with R₇;
each R₆ is independently alkyl, CF₃, OH, Oalkyl, OCOalkyl, CONH2, CN, halogen, NH₂, NO₂, NHCHO, NHCONH₂, NHSO₂alkyl, CONH₂, SOMe, SO₂NH₂, Salkyl, CH₂SO₂alkyl or OCONalkyl₂;
R₇ is R₈ or (CH₂)ₙOR₈, R₉, CF₃, OH, OR₉, OCOR₉, COR₉, COOR₉, CONH₂, CH₂CONH₂, CN, halogen, NH₂, NO₂, NHCHO, NHCONH₂, NHCONHR₇, NHCON(R₉)₂, NHCOR₉, NHCOaryl, NHSO₂Me, CONH₂, SMe, SOMe Or SO₂NH₂;
R₈ is (CH₂)ₙOR₉, (CH)ₙOR₉, (CH₂)ₙCOOR₉ or (CH₂)ₙCOaryl;
R₉ is alkyl or cycloalkyl; and
n is 1 to 4;
or a salt thereof.

2. The method according to claim 1, wherein the condition is a chronic degenerative disease.

3. The method according to claim 1, wherein the condition is a chronic demyelinating disease.

4. The method according to claim 1, wherein the condition is a respiratory disease.

5. The method according to claim 1, wherein the condition is an inflammatory bowel disease (IBD).

6. The method according to claim 1, wherein the condition is a dermatological condition.

7. The method according to claim 1, wherein the condition is a dental disease.

8. The method according to claim 1, wherein the condition is diabetic nephropathy, lupus nephritis, IgA nephropathy or glomerulonephritis.

9. The method according to claim 1, wherein the condition is systemic lupus erythematosus (SLE).

10. The method according to claim 1, wherein the condition is graft vs host disease.

11. The method according to claim 1, wherein the condition is a pain condition.

12. The method according to claim 11, wherein the pain condition is chronic pain.

13. The method according to claim 11, wherein the pain condition is acute pain.

14. The method according to claim 11, wherein the pain condition is neuropathic pain.

15. The method, according to claim 1, wherein
R₁ is CHR₄—OR₅, or aryl or heteroaryl optionally substituted with one or more groups R₆;
each R₆ is independently alkyl, CF₃, OH, Oalkyl, OCOalkyl, CONH₂, CN, halogen, NH₂, NO₂, NHCHO, NHCONH₂, NHSO₂alkyl, CONH₂, SOMe, Salkyl, CH₂S0₂alkyl or OCONalkyl₂; and
R₇ is R₈ or (CH₂)ₙOR₈, R₉, CF₃, OH, OR₉, OCOR₉, COR₉, COOR₉, CONH₂, CH₂CONH₂, CN, halogen, NH₂, NO₂, NHCHO, NHCONH₂, NHCONHR₇, NHCON(R₉)₂, NHCOR₉, NHCOaryl, NHSO₂Me, CONH₂, SMe or SOMe.

16. The method according to claim 15, wherein R₁ is aryl or heteroaryl.

17. The method according to claim 15, wherein R₁ is CHR₄—OR₅.

18. The method, according to claim 1, wherein the compound is (S)-clenbuterol, (S)-mabuterol, (S)(R)-rimiterol or (S)(S)-rimiterol.

19. The method according to claim 1, wherein the compound is atenolol, bucumolol or procaterol.

20. The method according to claim 1, wherein the patient is also administered another therapeutic agent selected from corticosteroids, cytotoxics, antibiotics, immunosupressants, non-steroidal anti-inflammatory drugs, narcotic analgesics, local anaesthetics, NMDA antagonists, neuroleptics, anti-convulsants, anti-spasmodics, anti-depressants and muscle relaxants.

21. The method according to claim 20, wherein the compound (I) and said another agent are provided in combination.

22. The method, according to claim 2, wherein the chronic degenerative disease is rheumatoid arthritis, osteoarthritis or osteoporosis.

23. The method, according to claim 3, wherein the condition is multiple sclerosis.

24. The method, according to claim 4, wherein the condition is asthma or chronic obstructive pulmonary disease.

25. The method, according to claim 5, wherein the condition is ulcerative colitis or Crohn's disease.

26. The method, according to claim 6, wherein the condition is scleroderma or atopic dermatitis.

27. The method, according to claim 7, wherein the condition is periodontal disease or gingivitis.

28. The method, according to claim 12, wherein the condition is chronic back pain, malignant pain, chronic headache or arthritic pain.

29. The method, according to claim 13, wherein the condition is postoperative pain, post-traumatic pain or acute disease-induced pain.
